(19) United States
(12) Patent Application Publication
(10) Pub. No.: US 2006/0089597 A1
(43) Pub. Date: Apr. 27, 2006

(54) Hypodermic syringe with multi-gauge needle exchangeability

(76) Inventor: Edward F. Allard, Springfield, VA (US)

Correspondence Address: Edward F. Allard, 7830 Greeley Blvd, Springfield, VA, 22152, US

(21) Appl. No.: 10/970,856

(22) Filed: Oct. 21, 2004

(51) Int. Cl. A61M 5/00; A61M 5/31

(52) U.S. Cl. 604/110; 604/240

(57) ABSTRACT

An improvement in a safety syringe having a retractable needle assembly which is automatically retracted into the plunger of the syringe upon the evacuation of all the fluid in the syringe and upon the end of the plunger triggering the release of a locking mechanism which allows the pent up energy of a biased spring to force the needle assembly into the barrel of the plunger for safe storage, wherein the needle assembly is adapted to receive interchangeable gauge needles.

### BACKGROUND OF THE INVENTION

1. Field of the Invention

The present invention generally relates to hypodermic syringes which may, after use, be retracted into the syringe to protect the user from the inadvertent exposure to possible contaminated fluid from the needle, and more specifically, to a syringe having an automatically retractable needle assembly which may be withdrawn into the syringe housing after use and which is adaptable to accept different gauge needles.

2. Description of the Prior Art

Automatically retractable needles have become the most desirable approach to help alleviate the rampant spread of diseases from the inadvertent exposure to contaminated fluids from a used syringe, however, the necessity to have readily available syringes with different gauge needles generates a storage problem, a procurement problem and a storage retrievable problem. While automatically retractable syringes are known in the prior art and the interchangeability of different gauge needles has been attempted in various instances, no record has been found of successfully merging the two requirements. The instant technique for interchanging different gauge needles in a syringe having an automatically retractable needle, such as that described in U.S. Pat. No. 6,050,974, actually uses the syringe therein described as a typical syringe body which may be used as a standard syringe body with a slight modification of the needle assembly for effecting the interchangeability of different gauge needles to suit the occasion.

### SUMMARY OF THE INVENTION

The general purpose of this invention is to provide a simple and easy design and technique for interchanging different gauge needles while using one standard hypodermic syringe. In the instant case, a hypodermic syringe having a needle assembly which may be retracted into the syringe housing allows for inserting any desired gauge needle into the needle assembly prior to use without interfering with the retractability of the needle assembly.

### BRIEF DESCRIPTION OF DRAWINGS

FIG. 1 shows a cut-away view of the prior art hypodermic syringe in U.S. Pat. No. 6,050,974.

FIG. 2 shows a cut-away view of the improved needle assembly of the syringe in FIG. 1 having means for interchanging different gauge needles.

### DESCRIPTION OF THE PREFERRED EMBODIMENTS

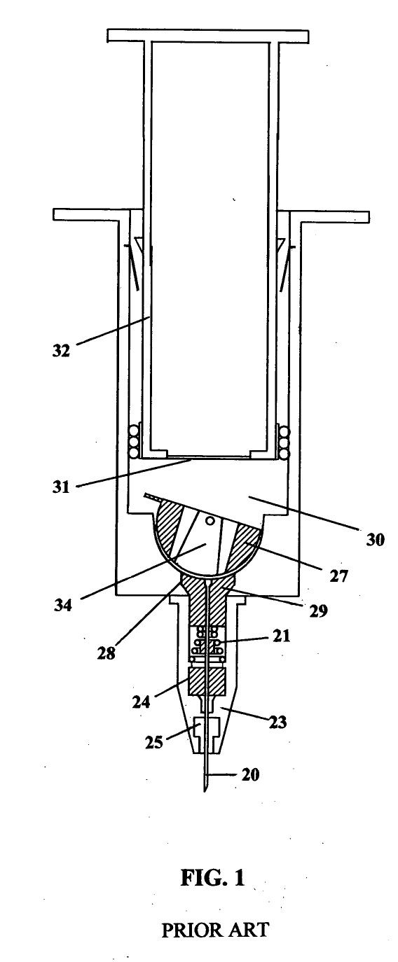

Looking now at the prior art in FIG. 1, one sees a detailed view of the quasi-cylindrical rotatable lock mechanism (27), having a filler core (34) in the center thereof, and where the lock mechanism is normally tilted at a slight angle, such that the central axis of the rotatable lock is skewed relative to the central axis of syringe body. In order to maintain the needle assembly, which comprises the needle head holder holder (29), fused to needle (20) which is housed within a housing tip (23), fused to the syringe housing (30) on the distal end of the syringe, in a state of equilibrium, at least some portion of the arcuate surface of the non-filler core portion of the surface of the rotatable lock (27) is always in contact with the head of the needle head holder (29), until all the medication has been expelled from the syringe and the needle assembly is properly aligned to allow retraction of the needle assembly into the plunger. The spring (21) applies an upward force to the needle head holder (29), which is seated in a recess (28) in the body of the syringe housing (30) and the rotatable lock mechanism (27) applies a restraining force on the opposite end of the needle head holder (29) from the side on which the upward force of the spring (21) is applied to maintain the needle assembly in state of equilibrium, until the plunger (32) reaches the bottom of the barrel of the syringe (30), whereupon the plunger makes contact with the rotatable lock mechanism and rotates the lock, such that the central axis of the rotatable lock (27) becomes aligned with the central axis of the syringe housing (30), allowing the needle head holder (29) to force a filler core (34), in the center of the rotatable lock mechanism (27), to be released, thereby allowing the core (34), needle assembly and spring to be ejected into the barrel of the plunger (32), as diaphragm (31) is ruptured by the upward force of the core (34). The needle assembly is thusly forced into the barrel of the plunger (32) by the force of the spring (21) whereby the barrel of the plunger becomes a safe storage container for the needle assembly to prevent any further contact therewith by the user.

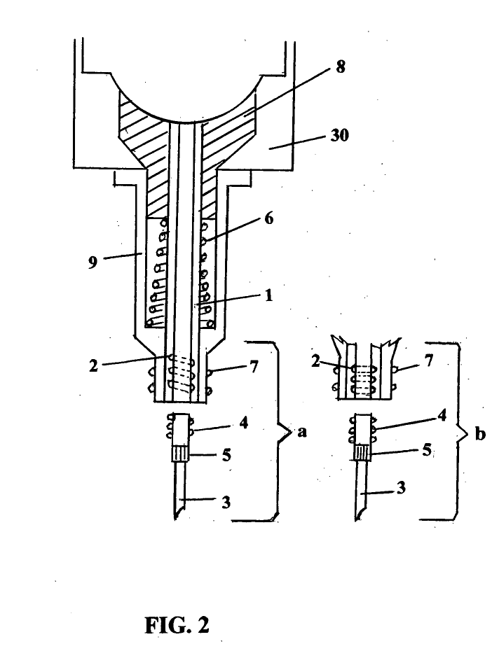

Looking now at the needle assembly of FIG. 2, one notes that the needle (20) and the needle head holder (29) of FIG. 1 has been replaced with a needle head extension holder (8) having a needle extension tube (1) extending through and fused to the center of the needle head extension holder (8), into which a needle (3), of any desired gauge, may be attached. The needle head extension holder (8) may be made from any suitable material compatible with the other materials used in the syringe and other components, but is preferably fabricated from rubber or plastic. The needle extension tube (1) is preferably made of plastic and the distal end of the tube is here shown as having female threads on the inside thereof (see “a” of FIG. 2) for accepting a male threaded needle head, but should a snap-in needle be desired, (see inset view “b” of FIG. 2) then the end of the tube could easily be grooved to contain two or more circular grooves to accept a needle having two or more rows of circumferential ridges extending from the outside end of the needle to form a male connection for connecting the ridges of the male headed needle into the female grooves within the inner surface of the needle extension tube (1). The specific means of attachment of the needle (3) to the extension tube (1) is shown as mating male threads (4) on the outside end of the needle (3) with female threads (2) on the inside end of the extension tube (1). A plastic sleeve, fused to needle (3), has the male threads (4) thereon, along with a knurled area for providing a firm grip on the needle when inserting and twisting the needle into the tube. This needle may be of any desired gauge and with it being much shorter in length than a standard needle in a standard syringe, a considerable savings will be recognized by the use of less metal in the fabrication. Also note that these small interchangeable needles may be stored in much less space than that of a series of different gauge standard syringes, thus realizing further savings in storage space. Note also that the housing tip (23) of the prior art device in FIG. 1 is redesigned in the improvement shown in FIG. 2, by eliminating the syringe seal (24) and the fluid collecting chamber (25). The housing tip (9) may be attached to the syringe housing (30) by any of several known techniques, such as by gluing, by using snap-on connectors or by heat fusion. Upon use of the syringe with the improved interchangeable gauge needles, retraction of the needle assembly functions the same as described above, relative to the release of the biased spring (6) and the retraction of the needle assembly, just as in the prior art device of FIG. 1. Outward extending extrusions (7) are shown on the outer surface of the lower end of the housing tip to function as a guide in allowing for the application of a safety cap (not shown) to the end of the housing tip (9), after retraction of the needle assembly.

Having thus described my invention, what I claim as new and desire to secure by Letters Patent is as follows:

1. In a hypodermic syringe having a retractable spring loaded needle assembly held in a biased state by a rotatable member further having a dislodgable filler core extending through a hole in the center thereof, such that upon releasing the biased needle assembly, the spring expands, forcing the needle assembly to dislodge the filler core from the hole in the rotatable member, such that the needle assembly is forced into the plunger of the syringe for safe storage, an improvement comprising the needle assembly having a needle extension tube and a needle head extension holder, wherein the needle extension head holder is firmly attached to one end of the needle extension tube with the other end of the needle extension tube being adapted to couple interchangeable gauge hypodermic needles.

2. The improvement of claim 1, wherein the end of the needle extension tube is adapted to threadably engage one end of a threaded hypodermic needle.

3. The improvement of claim 2, wherein the end of the needle extension tube adapted to couple a hypodermic needle thereto has at least two circumferential grooves around the inside surface thereof for mating with ridges on one end of the outside of a hypodermic needle.

4. The improvement of claim 2, wherein the needle assembly further includes a needle, having a sleeve with threads moulded to one end thereof and threadably engaged with one end of the needle extension tube.

5. The improvement of claim 3, wherein the needle assembly further includes a hypodermic needle, having a sleeve with at least two circumferential ridges around the outer surface of one end thereof mated with the circumferential grooves around the inner surface of the needle extension tube.
